SEQUENCE LISTING (10 sequences)

SEQ ID NO: 1 - DNA, 2083 bp, Xerophyta viscosa
actgtctgggtagctggcaatatagagacgtaagaaattcatggatcatcaccctaattcggtctttcactcattttatcctagacctgactaaaaaacttggtcagagttttacttatttaaaaaaaagaggacttcatggcatccatgtgcaggtacagctcccagaaaaaaaaagcatgaaacacgacaggatcaatagcattcgatctgaaacaaaaggttggagctcaagactttctccaaaatattaagatgatccaaagaattaccccaagatatccaacgtataccaatgtgtataccgaaagtaagaaagttcacgtgcattctttgatttttctcccgagtgttcttttctgaaatgagtaaataagactagaataagagctaatgtattttttttctaaaaaaaagttgaatgtggatacaatatgattatacattcattagctattttaagtatattctattttttttccccccaaaagaacacaaatgtgttccgtcactttccatggggcaaattacaacttaggctttatcttagttggtatgatcttaattttattatactttaaacaacttatcgctaataattttgttttgatttatgcgccaattgtaaatataaatcggataaatttgaacattaatacttttagtcagtttcaaaaaagaaaaagataactatgacgttagagtttggaatccagtcaaatggaacttattttttagttcatcagaatcaacttgatgagattttttgtactagacaatcatcttgaatgatagataggggacttaccaatcagccccccatattttgaaactttcaacgcgcccctcaacatttttctctttcaacgcgcccttccatcacttttctcttttcatcgcccctccctcaactttttggtcggacggaaatacctctatatatatttcatcatttcgacccccaaaacacctatttagaagtattttttggaaaaaattttaacatgaaagttttagatcttgatgagatctacaatttttatgttgaaagtttttccaaaaaatacttttagatagatattttggaactccgaagtgttaatgggtcgacccgctaacttgcggaaatagaaaaacatcaagatctacaacttttatgttgaaagttttttcaaaaaatacttctagatagatatttgatttgtaattttaatgttgaaagttttttcaaaaaatacttctagatagatactttggggctccggagtgttaacaagtataggaatatttttgtctgcaaaaaattaatttttcggacaagaggccgatcagtaaggaatctggtcggaggggctgttcggcaatataagttcagataggagaactgatcggatatttttccttaatttaattccaatttgatactattaagaatgaaaacatcctaataattgtgaccactttatagcaccacatttattttaatttaaatcttttaaatcttagaattggacagggtgcttatgataacaaacttgttcctatcaacaactgcatgttagacagcgccgaatttacagtcctactgggcgccacttttcaacccacatcatcaagatgaacaccacgttatcttcatccgctccaaccacatggtccagcgccactggccaagaccgccagccagccaggccatccaacgtggtgcattttctaacactccacgttcgctgtacggcattatttctccagccagaaagaccgagacagcgacgctgttgggcgggcccgcggcctgctctctctgcttccccatgagattcacgggcatcgctcctcgctcgtgcctacgcccgcgcccgcgcccgaccgcgccggtccacgtgacgtggcgcagcaatcgttcttactaggcgcttgcacgtgtcgttcgcatgcgaagcgtccacactgccaacgacctccttaaatatccttgtgatattcgccttacgatctcacacttcgcacgcaaaggccagtcgcagatttgggttgaatttgctgcgttttggcagattttgagcgagagatattagggaag

SEQ ID NO: 2 - DNA, 1577 bp, Xerophyta viscosa
cgtcactttccatggggcaaattacaacttaggctttatcttagttggtatgatcttaattttattatactttaaacaacttatcgctaataattttgttttgatttatgcgccaattgtaaatataaatcggataaatttgaacattaatacttttagtcagtttcaaaaaagaaaaagataactatgacgttagagtttggaatccagtcaaatggaacttattttttagttcatcagaatcaacttgatgagattttttgtactagacaatcatcttgaatgatagataggggacttaccaatcagccccccatattttgaaactttcaacgcgcccctcaacatttttctctttcaacgcgcccttccatcacttttctcttttcatcgcccctccctcaactttttggtcggacggaaatacctctatatatatttcatcatttcgacccccaaaacacctatttagaagtattttttggaaaaaattttaacatgaaagttttagatcttgatgagatctacaatttttatgttgaaagtttttccaaaaaatacttttagatagatattttggaactccgaagtgttaatgggtcgacccgctaacttgcggaaatagaaaaacatcaagatctacaacttttatgttgaaagttttttcaaaaaatacttctagatagatatttgatttgtaattttaatgttgaaagttttttcaaaaaatacttctagatagatactttggggctccggagtgttaacaagtataggaatatttttgtctgcaaaaaattaatttttcggacaagaggccgatcagtaaggaatctggtcggaggggctgttcggcaatataagttcagataggagaactgatcggatatttttccttaatttaattccaatttgatactattaagaatgaaaacatcctaataattgtgaccactttatagcaccacatttattttaatttaaatcttttaaatcttagaattggacagggtgcttatgataacaaacttgttcctatcaacaactgcatgttagacagcgccgaatttacagtcctactgggcgccacttttcaacccacatcatcaagatgaacaccacgttatcttcatccgctccaaccacatggtccagcgccactggccaagaccgccagccagccaggccatccaacgtggtgcattttctaacactccacgttcgctgtacggcattatttctccagccagaaagaccgagacagcgacgctgttgggcgggcccgcggcctgctctctctgcttccccatgagattcacgggcatcgctcctcgctcgtgcctacgcccgcgcccgcgcccgaccgcgccggtccacgtgacgtggcgcagcaatcgttcttactaggcgcttgcacgtgtcgttcgcatgcgaagcgtccacactgccaacgacctccttaaatatccttgtgatattcgccttacgatctcacacttcgcacgcaaaggccagtcgcagatttgggttgaatttgctgcgttttggcagattttgagcgagagatattagggaag

SEQ ID NO: 3 - DNA, 1127 bp, Xerophyta viscosa
gacccccaaaacacctatttagaagtattttttggaaaaaattttaacatgaaagttttagatcttgatgagatctacaatttttatgttgaaagtttttccaaaaaatacttttagatagatattttggaactccgaagtgttaatgggtcgacccgctaacttgcggaaatagaaaaacatcaagatctacaacttttatgttgaaagttttttcaaaaaatacttctagatagatatttgatttgtaattttaatgttgaaagttttttcaaaaaatacttctagatagatactttggggctccggagtgttaacaagtataggaatatttttgtctgcaaaaaattaatttttcggacaagaggccgatcagtaaggaatctggtcggaggggctgttcggcaatataagttcagataggagaactgatcggatatttttccttaatttaattccaatttgatactattaagaatgaaaacatcctaataattgtgaccactttatagcaccacatttattttaatttaaatcttttaaatcttagaattggacagggtgcttatgataacaaacttgttcctatcaacaactgcatgttagacagcgccgaatttacagtcctactgggcgccacttttcaacccacatcatcaagatgaacaccacgttatcttcatccgctccaaccacatggtccagcgccactggccaagaccgccagccagccaggccatccaacgtggtgcattttctaacactccacgttcgctgtacggcattatttctccagccagaaagaccgagacagcgacgctgttgggcgggcccgcggcctgctctctctgcttccccatgagattcacgggcatcgctcctcgctcgtgcctacgcccgcgcccgcgcccgaccgcgccggtccacgtgacgtggcgcagcaatcgttcttactaggcgcttgcacgtgtcgttcgcatgcgaagcgtccacactgccaacgacctccttaaatatccttgtgatattcgccttacgatctcacacttcgcacgcaaaggccagtcgcagatttgggttgaatttgctgcgttttggcagattttgagcgagagatattagggaag

SEQ ID NO: 4 - DNA, 2083 bp, Xerophyta viscosa
tgacagacccatcgaccgttatatctctgcattctttaagtacctagtagtgggattaagccagaaagtgagtaaaataggatctggactgattttttgaaccagtctcaaaatgaataaattttttttctcctgaagtaccgtaggtacacgtccatgtcgagggtctttttttttcgtactttgtgctgtcctagttatcgtaagctagactttgttttccaacctcgagttctgaaagaggttttataattctactaggtttcttaatggggttctataggttgcatatggttacacatatggctttcattctttcaagtgcacgtaagaaactaaaaagagggctcacaagaaaagactttactcatttattctgatcttattctcgattacataaaaaaaagattttttttcaacttacacctatgttatactaatatgtaagtaatcgataaaattcatataagataaaaaaaaggggggttttcttgtgtttacacaaggcagtgaaaggtaccccgtttaatgttgaatccgaaatagaatcaaccatactagaattaaaataatatgaaatttgttgaatagcgattattaaaacaaaactaaatacgcggttaacatttatatttagcctatttaaacttgtaattatgaaaatcagtcaaagttttttctttttctattgatactgcaatctcaaaccttaggtcagtttaccttgaataaaaaatcaagtagtcttagttgaactactctaaaaaacatgatctgttagtagaacttactatctatcccctgaatggttagtcggggggtataaaactttgaaagttgcgcggggagttgtaaaaagagaaagttgcgcgggaaggtagtgaaaagagaaaagtagcggggagggagttgaaaaaccagcctgcctttatggagatatatataaagtagtaaagctgggggttttgtggataaatcttcataaaaaaccttttttaaaattgtactttcaaaatctagaactactctagatgttaaaaatacaactttcaaaaaggttttttatgaaaatctatctataaaaccttgaggcttcacaattacccagctgggcgattgaacgcctttatctttttgtagttctagatgttgaaaatacaactttcaaaaaagttttttatgaagatctatctataaactaaacattaaaattacaactttcaaaaaagttttttatgaagatctatctatgaaaccccgaggcctcacaattgttcatatccttataaaaacagacgttttttaattaaaaagcctgttctccggctagtcattccttagaccagcctccccgacaagccgttatattcaagtctatcctcttgactagcctataaaaaggaattaaattaaggttaaactatgataattcttacttttgtaggattattaacactggtgaaatatcgtggtgtaaataaaattaaatttagaaaatttagaatcttaacctgtcccacgaatactattgtttgaacaaggatagttgttgacgtacaatctgtcgcggcttaaatgtcaggatgacccgcggtgaaaagttgggtgtagtagttctacttgtggtgcaatagaagtaggcgaggttggtgtaccaggtcgcggtgaccggttctggcggtcggtcggtccggtaggttgcaccacgtaaaagattgtgaggtgcaagcgacatgccgtaataaagaggtcggtctttctggctctgtcgctgcgacaacccgcccgggcgccggacgagagagacgaaggggtactctaagtgcccgtagcgaggagcgagcacggatgcgggcgcgggcgcgggctggcgcggccaggtgcactgcaccgcgtcgttagcaagaatgatccgcgaacgtgcacagcaagcgtacgcttcgcaggtgtgacggttgctggaggaatttataggaacactataagcggaatgctagagtgtgaagcgtgcgtttccggtcagcgtctaaacccaacttaaacgacgcaaaaccgtctaaaactcgctctctataatcccttc

SEQ ID NO: 5 - DNA, 1577 bp, Xerophyta viscosa
gcagtgaaaggtaccccgtttaatgttgaatccgaaatagaatcaaccatactagaattaaaataatatgaaatttgttgaatagcgattattaaaacaaaactaaatacgcggttaacatttatatttagcctatttaaacttgtaattatgaaaatcagtcaaagttttttctttttctattgatactgcaatctcaaaccttaggtcagtttaccttgaataaaaaatcaagtagtcttagttgaactactctaaaaaacatgatctgttagtagaacttactatctatcccctgaatggttagtcggggggtataaaactttgaaagttgcgcggggagttgtaaaaagagaaagttgcgcgggaaggtagtgaaaagagaaaagtagcggggagggagttgaaaaaccagcctgcctttatggagatatatataaagtagtaaagctgggggttttgtggataaatcttcataaaaaaccttttttaaaattgtactttcaaaatctagaactactctagatgttaaaaatacaactttcaaaaaggttttttatgaaaatctatctataaaaccttgaggcttcacaattacccagctgggcgattgaacgcctttatctttttgtagttctagatgttgaaaatacaactttcaaaaaagttttttatgaagatctatctataaactaaacattaaaattacaactttcaaaaaagttttttatgaagatctatctatgaaaccccgaggcctcacaattgttcatatccttataaaaacagacgttttttaattaaaaagcctgttctccggctagtcattccttagaccagcctccccgacaagccgttatattcaagtctatcctcttgactagcctataaaaaggaattaaattaaggttaaactatgataattcttacttttgtaggattattaacactggtgaaatatcgtggtgtaaataaaattaaatttagaaaatttagaatcttaacctgtcccacgaatactattgtttgaacaaggatagttgttgacgtacaatctgtcgcggcttaaatgtcaggatgacccgcggtgaaaagttgggtgtagtagttctacttgtggtgcaatagaagtaggcgaggttggtgtaccaggtcgcggtgaccggttctggcggtcggtcggtccggtaggttgcaccacgtaaaagattgtgaggtgcaagcgacatgccgtaataaagaggtcggtctttctggctctgtcgctgcgacaacccgcccgggcgccggacgagagagacgaaggggtactctaagtgcccgtagcgaggagcgagcacggatgcgggcgcgggcgcgggctggcgcggccaggtgcactgcaccgcgtcgttagcaagaatgatccgcgaacgtgcacagcaagcgtacgcttcgcaggtgtgacggttgctggaggaatttataggaacactataagcggaatgctagagtgtgaagcgtgcgtttccggtcagcgtctaaacccaacttaaacgacgcaaaaccgtctaaaactcgctctctataatcccttc

SEQ ID NO: 6 - DNA, 1127 bp, Xerophyta viscosa
ctgggggttttgtggataaatcttcataaaaaaccttttttaaaattgtactttcaaaatctagaactactctagatgttaaaaatacaactttcaaaaaggttttttatgaaaatctatctataaaaccttgaggcttcacaattacccagctgggcgattgaacgcctttatctttttgtagttctagatgttgaaaatacaactttcaaaaaagttttttatgaagatctatctataaactaaacattaaaattacaactttcaaaaaagttttttatgaagatctatctatgaaaccccgaggcctcacaattgttcatatccttataaaaacagacgttttttaattaaaaagcctgttctccggctagtcattccttagaccagcctccccgacaagccgttatattcaagtctatcctcttgactagcctataaaaaggaattaaattaaggttaaactatgataattcttacttttgtaggattattaacactggtgaaatatcgtggtgtaaataaaattaaatttagaaaatttagaatcttaacctgtcccacgaatactattgtttgaacaaggatagttgttgacgtacaatctgtcgcggcttaaatgtcaggatgacccgcggtgaaaagttgggtgtagtagttctacttgtggtgcaatagaagtaggcgaggttggtgtaccaggtcgcggtgaccggttctggcggtcggtcggtccggtaggttgcaccacgtaaaagattgtgaggtgcaagcgacatgccgtaataaagaggtcggtctttctggctctgtcgctgcgacaacccgcccgggcgccggacgagagagacgaaggggtactctaagtgcccgtagcgaggagcgagcacggatgcgggcgcgggcgcgggctggcgcggccaggtgcactgcaccgcgtcgttagcaagaatgatccgcgaacgtgcacagcaagcgtacgcttcgcaggtgtgacggttgctggaggaatttataggaacactataagcggaatgctagagtgtgaagcgtgcgtttccggtcagcgtctaaacccaacttaaacgacgcaaaaccgtctaaaactcgctctctataatcccttc

SEQ ID NO: 7 - DNA, 2083 bp, Xerophyta viscosa
cttccctaatatctctcgctcaaaatctgccaaaacgcagcaaattcaacccaaatctgcgactggcctttgcgtgcgaagtgtgagatcgtaaggcgaatatcacaaggatatttaaggaggtcgttggcagtgtggacgcttcgcatgcgaacgacacgtgcaagcgcctagtaagaacgattgctgcgccacgtcacgtggaccggcgcggtcgggcgcgggcgcgggcgtaggcacgagcgaggagcgatgcccgtgaatctcatggggaagcagagagagcaggccgcgggcccgcccaacagcgtcgctgtctcggtctttctggctggagaaataatgccgtacagcgaacgtggagtgttagaaaatgcaccacgttggatggcctggctggctggcggtcttggccagtggcgctggaccatgtggttggagcggatgaagataacgtggtgttcatcttgatgatgtgggttgaaaagtggcgcccagtaggactgtaaattcggcgctgtctaacatgcagttgttgataggaacaagtttgttatcataagcaccctgtccaattctaagatttaaaagatttaaattaaaataaatgtggtgctataaagtggtcacaattattaggatgttttcattcttaatagtatcaaattggaattaaattaaggaaaaatatccgatcagttctcctatctgaacttatattgccgaacagcccctccgaccagattccttactgatcggcctcttgtccgaaaaattaattttttgcagacaaaaatattcctatacttgttaacactccggagccccaaagtatctatctagaagtattttttgaaaaaactttcaacattaaaattacaaatcaaatatctatctagaagtattttttgaaaaaactttcaacataaaagttgtagatcttgatgtttttctatttccgcaagttagcgggtcgacccattaacacttcggagttccaaaatatctatctaaaagtattttttggaaaaactttcaacataaaaattgtagatctcatcaagatctaaaactttcatgttaaaattttttccaaaaaatacttctaaataggtgttttgggggtcgaaatgatgaaatatatatagaggtatttccgtccgaccaaaaagttgagggaggggcgatgaaaagagaaaagtgatggaagggcgcgttgaaagagaaaaatgttgaggggcgcgttgaaagtttcaaaatatggggggctgattggtaagtcccctatctatcattcaagatgattgtctagtacaaaaaatctcatcaagttgattctgatgaactaaaaaataagttccatttgactggattccaaactctaacgtcatagttatctttttcttttttgaaactgactaaaagtattaatgttcaaatttatccgatttatatttacaattggcgcataaatcaaaacaaaattattagcgataagttgtttaaagtataataaaattaagatcataccaactaagataaagcctaagttgtaatttgccccatggaaagtgacggaacacatttgtgttcttttggggggaaaaaaaatagaatatacttaaaatagctaatgaatgtataatcatattgtatccacattcaacttttttttagaaaaaaaatacattagctcttattctagtcttatttactcatttcagaaaagaacactcgggagaaaaatcaaagaatgcacgtgaactttcttactttcggtatacacattggtatacgttggatatcttggggtaattctttggatcatcttaatattttggagaaagtcttgagctccaaccttttgtttcagatcgaatgctattgatcctgtcgtgtttcatgctttttttttctgggagctgtacctgcacatggatgccatgaagtcctcttttttttaaataagtaaaactctgaccaagttttttagtcaggtctaggataaaatgagtgaaagaccgaattagggtgatgatccatgaatttcttacgtctctatattgccagctacccagacagt

SEQ ID NO: 8 - DNA, 1577 bp, Xerophyta viscosa
cttccctaatatctctcgctcaaaatctgccaaaacgcagcaaattcaacccaaatctgcgactggcctttgcgtgcgaagtgtgagatcgtaaggcgaatatcacaaggatatttaaggaggtcgttggcagtgtggacgcttcgcatgcgaacgacacgtgcaagcgcctagtaagaacgattgctgcgccacgtcacgtggaccggcgcggtcgggcgcgggcgcgggcgtaggcacgagcgaggagcgatgcccgtgaatctcatggggaagcagagagagcaggccgcgggcccgcccaacagcgtcgctgtctcggtctttctggctggagaaataatgccgtacagcgaacgtggagtgttagaaaatgcaccacgttggatggcctggctggctggcggtcttggccagtggcgctggaccatgtggttggagcggatgaagataacgtggtgttcatcttgatgatgtgggttgaaaagtggcgcccagtaggactgtaaattcggcgctgtctaacatgcagttgttgataggaacaagtttgttatcataagcaccctgtccaattctaagatttaaaagatttaaattaaaataaatgtggtgctataaagtggtcacaattattaggatgttttcattcttaatagtatcaaattggaattaaattaaggaaaaatatccgatcagttctcctatctgaacttatattgccgaacagcccctccgaccagattccttactgatcggcctcttgtccgaaaaattaattttttgcagacaaaaatattcctatacttgttaacactccggagccccaaagtatctatctagaagtattttttgaaaaaactttcaacattaaaattacaaatcaaatatctatctagaagtattttttgaaaaaactttcaacataaaagttgtagatcttgatgtttttctatttccgcaagttagcgggtcgacccattaacacttcggagttccaaaatatctatctaaaagtattttttggaaaaactttcaacataaaaattgtagatctcatcaagatctaaaactttcatgttaaaattttttccaaaaaatacttctaaataggtgttttgggggtcgaaatgatgaaatatatatagaggtatttccgtccgaccaaaaagttgagggaggggcgatgaaaagagaaaagtgatggaagggcgcgttgaaagagaaaaatgttgaggggcgcgttgaaagtttcaaaatatggggggctgattggtaagtcccctatctatcattcaagatgattgtctagtacaaaaaatctcatcaagttgattctgatgaactaaaaaataagttccatttgactggattccaaactctaacgtcatagttatctttttcttttttgaaactgactaaaagtattaatgttcaaatttatccgatttatatttacaattggcgcataaatcaaaacaaaattattagcgataagttgtttaaagtataataaaattaagatcataccaactaagataaagcctaagttgtaatttgccccatggaaagtgacg

SEQ ID NO: 9 - DNA, 1127 bp, Xerophyta viscosa
cttccctaatatctctcgctcaaaatctgccaaaacgcagcaaattcaacccaaatctgcgactggcctttgcgtgcgaagtgtgagatcgtaaggcgaatatcacaaggatatttaaggaggtcgttggcagtgtggacgcttcgcatgcgaacgacacgtgcaagcgcctagtaagaacgattgctgcgccacgtcacgtggaccggcgcggtcgggcgcgggcgcgggcgtaggcacgagcgaggagcgatgcccgtgaatctcatggggaagcagagagagcaggccgcgggcccgcccaacagcgtcgctgtctcggtctttctggctggagaaataatgccgtacagcgaacgtggagtgttagaaaatgcaccacgttggatggcctggctggctggcggtcttggccagtggcgctggaccatgtggttggagcggatgaagataacgtggtgttcatcttgatgatgtgggttgaaaagtggcgcccagtaggactgtaaattcggcgctgtctaacatgcagttgttgataggaacaagtttgttatcataagcaccctgtccaattctaagatttaaaagatttaaattaaaataaatgtggtgctataaagtggtcacaattattaggatgttttcattcttaatagtatcaaattggaattaaattaaggaaaaatatccgatcagttctcctatctgaacttatattgccgaacagcccctccgaccagattccttactgatcggcctcttgtccgaaaaattaattttttgcagacaaaaatattcctatacttgttaacactccggagccccaaagtatctatctagaagtattttttgaaaaaactttcaacattaaaattacaaatcaaatatctatctagaagtattttttgaaaaaactttcaacataaaagttgtagatcttgatgtttttctatttccgcaagttagcgggtcgacccattaacacttcggagttccaaaatatctatctaaaagtattttttggaaaaactttcaacataaaaattgtagatctcatcaagatctaaaactttcatgttaaaattttttccaaaaaatacttctaaataggtgttttgggggtc

SEQ ID NO: 10 - DNA, 2083 bp, Xerophyta viscosa
actgtctgggtagctggcaatatagagacgtaaataattgtctgtaaatagggagaaattcatggatcatcaccctaattcggtctttcactcattttatcatagacctgactaaagaacttggtcagagtttttacttatttaaaataaagaggacttcatggcatccatgtgcaggtacagctcccagaaaaaaaagcatgaaacacgagaggatcaatagcattcgatctgaaacaaaaggttgcagctcaagactttctccaaaatattaagatgatccaaagaattaccccaagatatccaacgtataccaatgtgtataccgaaagtaagaaagttcacgtgcattctttgatttttctcccgagtgttcttttctgaaatgagtaaataagactagaataagagctaatgtattttttttctaaaaaaagttgaatgtggatacaatatgattatacattcattagctattttaagtatattctattttttttccccccaaaagaacacaaatgtgttccgtcactttccatggggcaaattacaacttaggctttatcttagttggtatgatcttaattttattatactttaaacaacttatcgctaataattttgttttgatttatgcgccaattgtaaataaatcggataaatttgaacattaatacttttagtcaaagtttcaaaaaagaaaaagataactatgacgttagagtttggaatccagtcaaatggaacttattttttagttcatcagaatcaacttgatgagattttttgtactagacaatcatcttgaatgatagataggggacttaccaatcagccccccatattttgaaactttcaacgcgccccccaatatttttctctttcaacgcgcccttccatcacttttctcttttcatcgcccctccctcaactttttggtcggacggaaatacctctatatatatttcatcatttcgacaccccaaaacacttatttagaagtattttttgaaaaaaattttaacatgaaagttttagaccttgatgagatctacaacttttatgttgaaagtttttccaaaaaatacttttagatagatattttgaaactccgaagtgttaatgggtcgacccgctaacttgcagaaatagaaaaacatcaagatctacaatttttatgttgaaagttttttaaaaaaatatagatagatatttgatttataattttaatattgaaagttttttcaaaaaatacttctagatagatattttgggactctggaatgttacaagtataggaatatttttgtctgtaaaaaattaatttttcagacaaagggctgatcagtaaggaatctggtcgggggagctgttcggcaatataagttcagataggagaactaatcggatatttttccttaatttaattccgatttgatactattaagaatgaaaacatcctaataattgtgaccactttatagcaccacatttattttaattttaatcttttaaatcttagaattggacagggtgcttatgatacaacttgttcctatcaacaactgcatgttagacagcgccgaatttacagtcctactgggcgccacttttcaacccacatcatcaagatgaacaccacgttatcttcatccgctccaaccacatggtccagcgccactggccaagaccgccagccagccaggccatccaacgtggtgcattttctaacactccacgttcgctgtacggcattatttctccagccagaaagaccgagacagcgacgctgttgggcgggcccgcggcctgctctctctgcttccccatgagattcacgggcatcgctcctcgctcgtgcctacgcgaccgcgccgatccacgtgacgtggcgcagcaatcgttcttactaggcgcttgcacgtgtcgttcgcatgcgaagcgtccacactgccaacgacctccttaaatatccttgtgatattcgccttacgatctcacacttcgcacgcaaaggccagtcgcagatttgggttgaatttgctgcgttttggcagattttgagcgagagatattagggaag

(19) United States
(12) Patent Application Publication
(10) Pub. No.: US 2010/0275327 A1
(43) Pub. Date: Oct. 28, 2010

(54) METHOD OF INCREASING ABIOTIC STRESS TOLERANCE IN PLANTS

(75) Inventors: Jennifer Ann Thomson, Cape Town (ZA); Jesse Machuka, Nairobi (KE); Revel Iyer, Estcourt (ZA); Richard Okoth, Nairobi (KE)

Correspondence Address: MCCARTER & ENGLISH, LLP WILMINGTON;BASIL S. KRIKELIS, Renaissance Centre, 405 N. King Street, 8th Floor, WILMINGTON, DE, 19801, US

(73) Assignees: University of Cape Town, Cape Town (ZA); The Maize Trust, Queenswood (ZA)

(21) Appl. No.: 12/773,463

(22) Filed: May 4, 2010

Related U.S. Application Data
(63) Continuation-in-part of application No. PCT/IB2008/054628, filed on Nov. 6, 2008.

(30) Foreign Application Priority Data
Nov. 6, 2007 (ZA) 2007/09551

(51) Int. Cl. A01H 5/00; C12N 15/82; C07H 21/04

(52) U.S. Cl. 800/278; 800/298; 800/320.1; 800/320; 800/320.2; 800/320.3; 800/317.3; 800/312; 800/322; 800/314; 800/306; 536/23.1

(57) ABSTRACT

A method of increasing abiotic stress tolerance in a plant is described, the method comprising transforming the plant with a vector comprising a nucleic acid which is at least 80% identical to SEQ I.D. NO: 10. Plants or plant parts transformed with these sequences are also described.

### CROSS REFERENCE TO RELATED APPLICATIONS

The present application claims the benefit of PCT/IB2008/054628, filed Nov. 6, 2008 and South African Application No. 2007/09551, filed Nov. 6, 2007, the disclosures of which are expressly incorporated herein by reference in their entireties.

### BACKGROUND OF THE INVENTION

The invention describes plant promoters which are useful, for example, in agricultural biotechnology. In particular, the promoters are inducible under abiotic stress conditions, and can be used to develop transgenic plants which can express genes to ameliorate some adverse effects of abiotic stress.

To express coding regions of given genes that are included in transformation vectors or in DNA constructs it is required that a promoter is engineered upstream of the coding sequence. Promoters can now be selected that either allow constitutive gene expression or limit gene expression to only specific cell types or in response to specific environmental stimuli. There is a need to generate drought tolerant transgenic plants that express transgenes only under stress conditions, since overproduction of such proteins when constitutively expressed may hamper the normal growth of plants. This would be especially valuable in economically valuable crops such as maize and tobacco.

Under constitutive gene expression there is gene expression all the time and at high levels. Examples of constitutive promoters include the Cauliflower Mosaic Virus promoter for 35S RNA and the maize ubiquitin promoter. It has been reported that constitutive expression of proteins under normal growth conditions sometimes hampers the normal growth of transgenic plants, resulting in smaller phenotypes as compared to wild-type plants. This unwanted dwarfing of the transformed plants may be due to the expression of the protein in higher amounts than normal and at stages when it is not needed.

Some genes encode products that are only required under special conditions related to developmental stages of the plant, environmental stress, or pathogen attack. These genes contain ‘inducible promoters’ that can be turned on quickly by an inducer agent and are active for a limited length of time before they are turned off again. In the absence of an inducer, the DNA sequences or genes will not be transcribed. The inducer can be a chemical agent, or a physiological stress directly imposed upon the plant such as cold, heat, salt, toxins, or through the action of a pathogen or disease agent. A number of promoters that are inducible by anaerobic stress, high temperature stress, low temperature stress and salt stress have been described. However, it has been found that most of the stress-inducible promoters have poor strength of expression when compared to constitutive promoters.

Due to the poor strength of expression of inducible promoters when compared to constitutive promoters there is a need for the development of stress-responsive promoters that allow increased expression, but without any negative impact on their induction patterns.

The applicant has identified a need to provide transgenic plants which can express genes to ameliorate the adverse effects of abiotic stress, and to this end has isolated a promoter for inducing expression of proteins in plants during abiotic stress conditions. Abiotic stresses are deleterious effects on plants caused by non biological agents such as drought, salinity, cold and extreme temperatures while biotic constraints are usually attributed to living systems like bacteria, fungi, viruses that cause diseases and insects that feed on plants.

### SUMMARY OF THE INVENTION

The invention provides an isolated nucleic acid comprising the nucleotide sequence which is at least 80% identical to:
(a) any one of SEQ I.D. NOs: 1-3 and 10;
(b) a fragment of any one of SEQ I.D. NOs: 1-3 and functions as a stress inducible promoter;
(c) a sequence which hybridizes under standard or stringent conditions to the nucleotide sequence of any one of SEQ I.D. NOs: 1-3 and functions as a stress inducible promoter;
(d) a complement of any one of SEQ I.D. NOs: 1-3 (SEQ I.D. NOs: 4-6);
(e) a sequence which hybridizes under standard or stringent conditions to the complement of any one of SEQ I.D. NOs: 1-3 and functions as a stress inducible promoter;
(f) a reverse complement of any one of SEQ I.D. NOs: 1-3 (SEQ I.D. NOs: 7-9); or
(g) a degenerate or allelic variant of any one of SEQ I.D. NOs: 1-3.

More preferably, the nucleotide sequence may have at least 90% identity to any one of (a)-(g) above.

More preferably, the nucleotide sequence may have at least 95% identity to any one of (a)-(g) above.

Even more preferably, the nucleotide sequence may comprise the nucleotide sequence of SEQ I.D. NO: 1 or 10 or a truncated sequence thereof, such as SEQ I.D. NO: 2 or SEQ I.D. NO: 3.

The nucleic acid may be a stress inducible plant promoter, and the stress may be an abiotic stress such as osmotic stress, dehydration stress, drought, salinity, cold, heat, dessication or extreme temperatures.

The nucleic acid may be derived from Xerophyta viscosa.

Hybridization may occur under stringent conditions that include a wash in 0.1 SSC at about 60 to about 65° C.

The invention extends to a plant vector including the promoter. The vector may also contain a gene downstream of the promoter.

The recombinant vector may be a T-derived plasmid construct of Agrobacterium tumefaciens.

The invention also extends to a host cell into which the vector has been transformed.

The host cell may be a plant cell.

The invention further extends to a transgenic plant or plant part transformed with the promoter. The transgenic plant or plant part may also be transformed with a gene under the control of the promoter. The gene may be any suitable gene, such as XvSap1, XvPrx2 or XvPer1.

The plant may be capable of expressing the gene in stress conditions.

The transgenic plant may be a monocotyledonous or dicotyledonous plant, such as maize, tobacco, sorghum, wheat, cassava, barley, oats, rye, sweet potatoes, soybean, alfalfa, tobacco, sunflower, cotton, or canola.

The transgenic plant parts may be selected from the group consisting of: cells, protoplasts, cell tissue cultures, callus, cell clumps, embryos, pollen, ovules, seeds, flowers, kernels, ears, cobs, leaves, husks, stalks, roots, root tips, anthers, seeds and silk.

The invention also provides a method for enhancing the stress tolerance of a plant by introducing a promoter comprising the nucleic acid of claim 1, and optionally also a gene under control of the promoter, into the plant.

### BRIEF DESCRIPTION OF THE FIGURES

FIG. 1: Nucleotide sequence of promoter XvPsap1 (2083 bp, SEQ I.D. NO: 1).

FIG. 2: Nucleotide sequence of promoter XvPsap2 (1577 bp, SEQ I.D. NO: 2).

FIG. 3: Nucleotide sequence of promoter XvPsap3 (1127 bp, SEQ I.D. NO: 3).

FIG. 4: Complementary sequence of promoter XvPsap1 (2083 bp, SEQ I.D. NO: 4).

FIG. 5: Complementary sequence of promoter XvPsap2 (1577 bp, SEQ I.D. NO: 5).

FIG. 6: Complementary sequence of promoter XvPsap3 (1127 bp, SEQ I.D. NO: 6).

FIG. 7: Reverse complementary sequence of promoter XvPsap1 (2083 bp, SEQ I.D. NO: 7).

FIG. 8: Reverse complementary sequence of promoter XvPsap2 (1577 bp, SEQ I.D. NO: 8).

FIG. 9: Reverse complementary sequence of promoter XvPsap3 (1127 bp, SEQ I.D. NO: 9).

FIG. 10: Expression profile curves of luc transcripts using qRT-PCR analyses. A: Expression profile in transgenic BMS cells following treatment with 200 mM NaCl for 72 hours. B: Expression profile following dehydration treatment of transgenic tobacco plants for 8 days. C: Expression profile following dehydration treatment of transgenic maize. Only the XvPsap1 construct was used to transform maize. Analysis of variance was conducted at P<0.05.

FIG. 11: Nucleotide sequence of promoter XvPsap1 (SEQ I.D. NO: 10).

### DETAILED DESCRIPTION OF THE INVENTION

Abiotic stress-inducible promoters for driving the expression of genes for the production of abiotic stress-resistant transgenic plants are described herein. Vectors containing the promoter and a gene operably associated with the promoter, and plants or plant parts transformed with the promoter and gene are also described.

Given the complexity of water use in land plants, especially during conditions that produce water deficit, the applicant is aware of only two promoters specifically associated with this aspect of plant physiology are commercially available. These promoters are derived from rice and Arabidopsis.

Promoters possess core and regulatory regions. The regulatory region is what differentiates promoters. These regulatory regions generally consist of negative regulatory elements, transcriptional enhancers, translational enhancers and other regulatory elements. Three lengths of the DNA promoter of the present invention have been synthesised (SEQ I.D. NOs: 1-3 and 10). These would differ in the regulatory regions. Consequently their activity and effectiveness might differ in different plants and under different stress conditions. Thus, each of the promoters may be recommended for use in different plants subjected to different stresses.

The DNA promoters of the present invention are derived from a ‘resurrection plant’, Xerophyta viscosa, which can withstand extreme desiccation, surviving for months with only 5% relative water content. Upon watering it can rehydrate within 80 hours.

The Xerophyta viscosa genome has not been sequenced. Consequently, the base sequence of the DNA promoter of the present invention would not be identifiable were it not isolated and cloned. Furthermore, the DNA promoters of the present invention are truncated forms of the naturally occurring promoter.

The DNA promoters of the present invention do not code for any known functional protein. Their biological function is linked to the expression of a protein, which increases the stress tolerance of Xerophyta viscosa. The promoters lack any significant identity to the two commercially available promoters or to any other known promoter sequence.

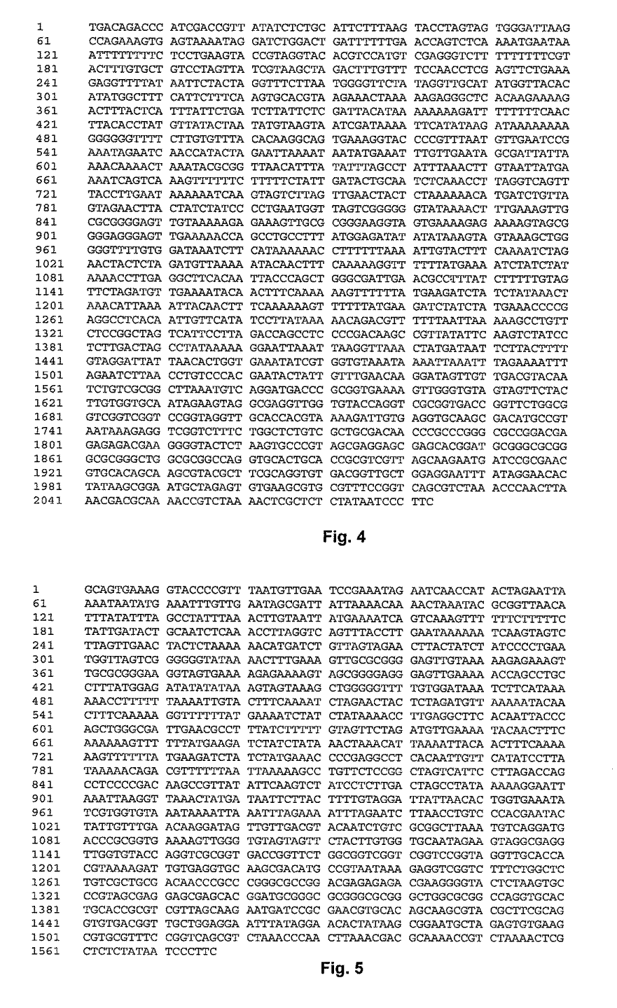

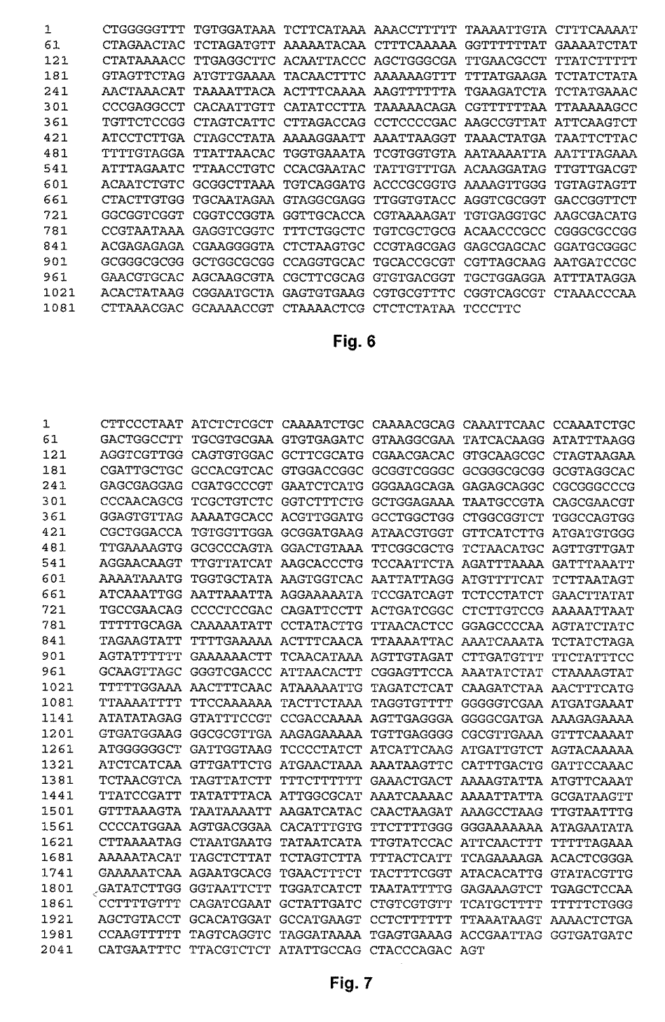

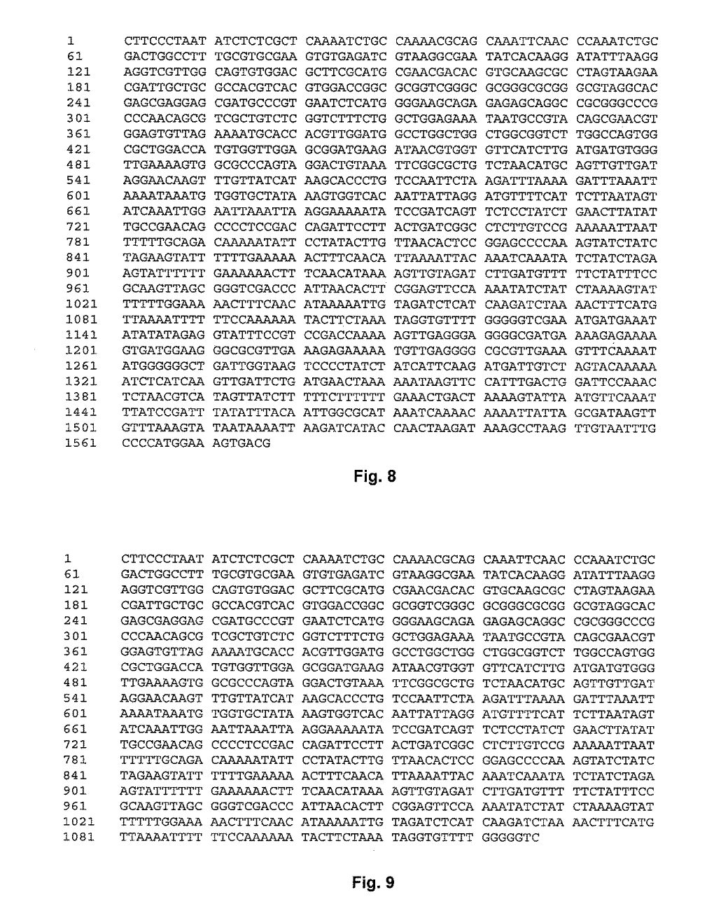

Although promoters having nucleotide sequences of SEQ I.D. NOs: 1-3 and 10 are described herein, it is envisaged that sequences which have 80% identity or more and which are functional promoters could also be used in the invention. Complementary sequences (SEQ I.D. NOs: 4-6), reverse complementary sequences (SEQ I.D. NOs: 7-9) and sequences which hybridise to these sequences, under standard or stringent conditions, could also be used, provided that they have promoter activity (FIGS. 4-9).

For example, hybridisation can be carried out for 18 h at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5×SSC).

The DNA promoters can be induced by environmental stresses, such as drought, without the application of chemicals. They allow for expression of a desired protein or mRNA within a short period of applying the stimulus.

The promoter may be inserted into a plant vector. Genes that could generate transgenic plants tolerant to abiotic stresses, such as XvSap1, XvPrx2 or XvPer1, can be cloned downstream of the promoter. When such a composite construct is introduced into a transgenic plant, the gene will only be expressed when the plant is subjected to an abiotic stress. Thus, plants susceptible to abiotic stress like maize, tobacco, sorghum, wheat, cassava and sweet potatoes that are physiologically normal under non-stressed conditions can be produced.

### Methodology

The invention is described in more detail by way of the following methodology, which is not to be construed as limiting in any way either the spirit or scope of the invention.

The splinkerette protocol was employed to obtain the upstream genomic sequence of XvSap1 (AY100455). This protocol was modified from Devon et al. (1995). Truncated versions of the promoter were generated by selective amplification of the full length promoter region.

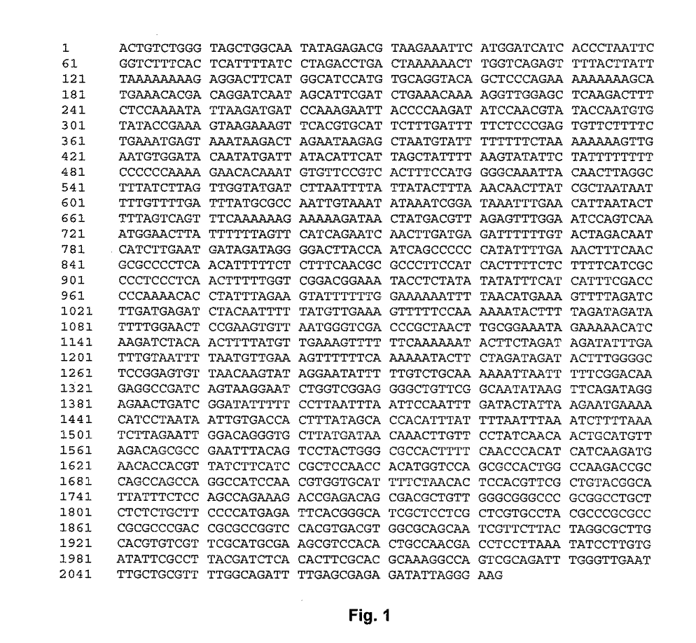

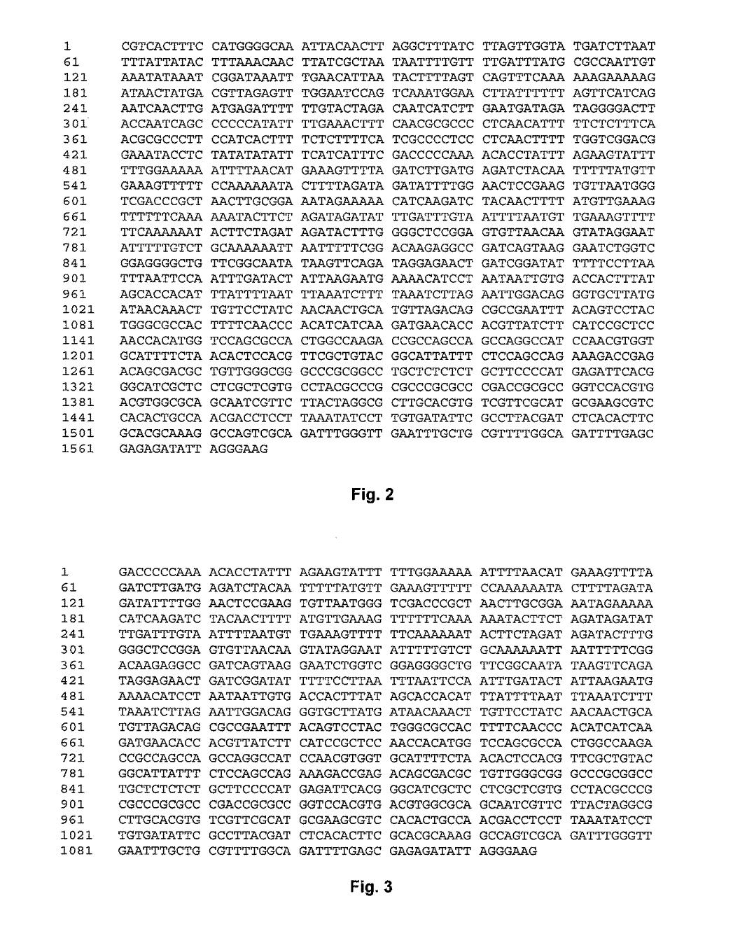

The gene expression performances of a drought-inducible promoter isolated from Xerophyta viscosa, XvPsap1 (SEQ I.D. NO. 1; FIG. 1 and SEQ I.D. NO: 10; FIG. 11) and its truncated fragments XvPsap2 (SEQ I.D. NO. 2; FIG. 2) and XvPsap3 (SEQ I.D. NO. 3; FIG. 3) were determined by Agrobacterium-mediated transformation of Nicotiana tabacum and Black Mexican Sweetcorn (BMS) cells with expression casettes containing the truncated promoters individually driving the expression of the luc reporter gene followed by a nos terminator. In addition, an expression cassette, containing XvPsap1 driving the expression of the luc reporter gene followed by a nos terminator, was transformed into maize.

To determine whether these promoters are stress-inducible, transcriptional analyses of luc in response to salt and dehydration stress were performed on transgenic cells and plants using quantitative real-time PCR (qPCR). Transgenic BMS cells were subjected to 200 mM NaCl salt stress. Transgenic tobacco and maize were exposed to dehydration treatment.

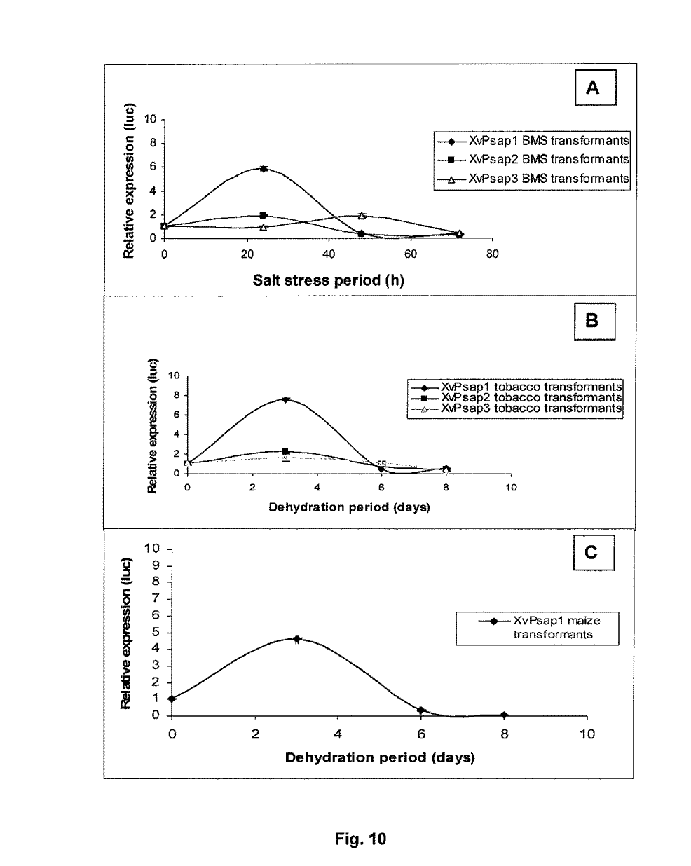

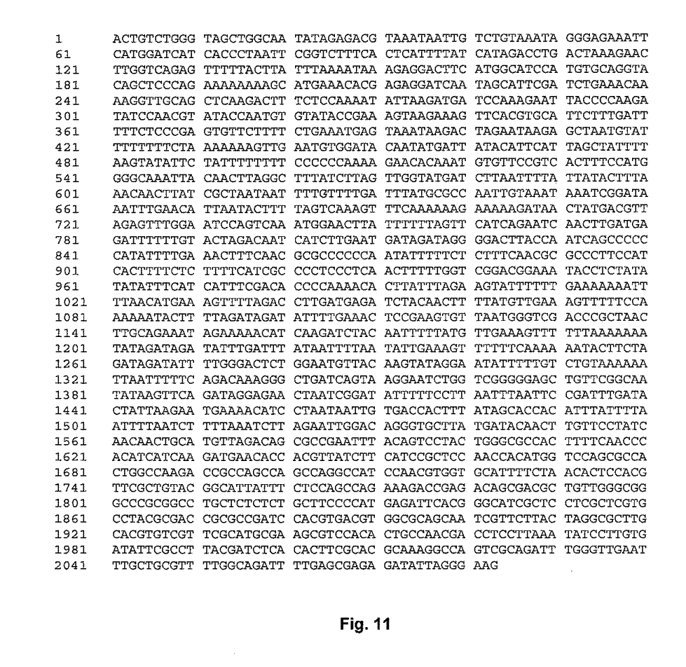

The qPCR analysis of the BMS cells indicated that luc mRNA was upregulated within 24 hours of salt stress for both XvPsap1 (5 fold; P<0.05) and XvPsap2 (1.9 fold; P<0.05) whereas with XvPsap3 (1.9 fold; P<0.05) upregulation occurred after 48 hours (FIG. 10A). A similar trend was observed with dehydrated tobacco, in which optimal expression levels were recorded 72 hours after initiating dehydration. The XvPsap1 activity was significant (7 fold; P<0.05) in transgenic tobacco whereas XvPsap2 and XvPsap3 displayed 2.2 and 1.6 fold increases, respectively (FIG. 10B). In dehydrated maize, XvPsap1 also displayed a significant peak in activity on the third day with a relative luc expression of 4 fold (FIG. 10C). Together, these results suggest that the XvPsap1 promoter is the most active and would be involved in the early responses to drought as it peaks shortly after transgenic plants are subjected to lack of water.

These findings are validated by Garwe et al. (2003, 2006) who isolated XvSap1 gene which XvPsap1 promoter naturally regulates in X. viscosa. They found that XvSap1 gene confers tolerance to dehydration, high temperatures and salinity in model plants. More recently, Iyer at al. (2007) reported a similar trend with the expression of XvSap1 gene in dehydrated X. viscosa. They noted an up-regulation of the XvSap1 mRNA at 60% relative water content. Thereafter, expression decreased but again increased at 15% RWC. This observation led them to conclude that XvSap1 could be involved in the initial and late stages of the protective response to dehydration.

It is anticipated that the gene expression of this stress tolerant maize line could be induced by environmental stress such as drought, without the need for the application of chemicals such that the stress tolerant maize will maintain its cellular membrane integrity and survive the adverse effects of reactive oxygen species (ROS) experienced during drought stress.

The following list of references are expressly incorporated herein by reference in their entireties.

Devon R S Porteous D J & Brokkes A J (1995) ‘Splinkerettes—improved vectorettes or greater efficiency in PCR walking’ Nucleic Acids Research 23:1644-1645.

Galun E & Breiman A (1996) ‘Transgenic plants’ Imperical College Press, USA.

Garwe D Thomson J A & Mundree S G (2003) ‘Molecular characterization of XVSAP1, a stress-responsive gene from the resurrection plant Xerophyta viscosa Baker’ Journal of Experimental Botany 54:191-201.

Garwe D Thomson J A & Mundree S G (2006) ‘XvSAP1 from Xerophyta viscosa improves osmotic-, salinity- and high-temperature-stress tolerance in Arabidopsis’ Biotechnology Journal 1:1-10.

Iyer R Mundree S G Rafudeen M S & Thomson J A (2007) ‘XvSap1, a desiccation tolerance associated gene with potential for crop improvement’ In Plant Desiccation Tolerance Jenks M A & Wood A J (eds.) Blackwell Publishing, UK 283-296.

Kasuga M Liu Q Miura S Yamaguchi-Shinozaki K & Shinozaki K (1999) ‘Improving plant drought, salt, and freezing tolerance by gene transfer of a single-inducible transcription factors’ Nature Biotechnology 17: 287-291.

Liu Q Kasuga M Sakuma Y Abe H Miura S Yamaguchi-Shinozaki K & Shinozaki K (1998) ‘Two transcriptional factors, DREB1 and DREB2, with an EREBP/AP2 DNA binding domain separate two cellular signal transduction pathways in drought and low temperature-responsive gene expression, respectively, in Arabidopsis’ Plant Cell 10:1391-1406.

Su J Shen Q Ho D T & Wu R (1998) Dehydration-stress-regulated transgene expression in stably transformed rice plants' Plant Physiology 117:913-922.

What is claimed is:

1. A method of increasing abiotic stress tolerance in a plant, the method comprising the step of transforming the plant with a vector comprising a nucleic acid having a nucleotide sequence which is at least 80% identical to SEQ I.D. NO: 10 to yield a transformed plant.

2. The method of claim 1, wherein the nucleic acid has the nucleotide sequence of SEQ I.D. NO: 10.

3. The method of claim 1, wherein the nucleic acid has the nucleotide sequence of SEQ I.D. NO: 1.

4. The method of claim 1, wherein the plant is an agricultural crop plant selected from the group consisting of maize, barley, oats, rye, tobacco, sorghum, wheat, cassava, rice, sweet potato, soybean, alfalfa, sunflower, cotton and canola.

5. The method of claim 1, wherein the transformed plant has increased tolerance to abiotic stress relative to an untransformed plant of the same plant species.

6. The method of claim 1, wherein the transformed plant has increased tolerance to cold stress relative to an untransformed plant of the same plant species.

7. The method of claim 1, wherein the transformed plant has increased tolerance to osmotic stress relative to an untransformed plant of the same plant species.

8. The method of claim 7, wherein the osmotic stress is selected from the group consisting of heat, drought, desiccation, freezing and high salt.

9. A transgenic plant, plant tissue or plant cell produced by the method of claim 1, which comprises a nucleic acid having a nucleotide sequence which is at least 80% identical to SEQ I.D. NO: 10 and which exhibits increased tolerance to abiotic stress relative to an untransformed plant of the same plant species.

10. The transgenic plant, plant tissue or plant cell of claim 9, which comprises a nucleic acid having a nucleotide sequence of SEQ I.D. NO: 10.

11. The transgenic plant, plant tissue or plant cell of claim 9, which is an agricultural crop plant or part thereof selected from the group consisting of maize, barley, oats, rye, tobacco, sorghum, wheat, cassava, rice, sweet potato, soybean, alfalfa, sunflower, cotton and canola.

12. The transgenic plant, plant tissue or plant cell of claim 9, which exhibits increased tolerance to cold stress relative to an untransformed plant of the same plant species.

13. The transgenic plant, plant tissue or plant cell of claim 9, which exhibits increased tolerance to osmotic stress relative to an untransformed plant of the same plant species.

14. Transgenic seeds produced by the transgenic plant of claim 9, wherein the seeds comprise a nucleic acid having a nucleotide sequence which is at least 80% identical to SEQ I.D. NO: 10.

15. Transgenic seeds of claim 14, which comprise a nucleic acid having a nucleotide sequence of SEQ I.D. NO: 10.

16. Transgenic seeds of claim 14, which are seeds of an agricultural crop plant selected from the group consisting of maize, barley, oats, rye, tobacco, sorghum, wheat, cassava, rice, sweet potato, soybean, alfalfa, sunflower, cotton and canola.

17. A stress-inducible promoter comprising a nucleic acid having a nucleotide sequence that is at least 80% identical to SEQ I.D. NO: 10 and which is capable of enhancing the abiotic stress tolerance of a plant that has been transformed with the nucleic acid.

18. The promoter of claim 17, which comprises a nucleic acid having a nucleotide sequence of SEQ I.D. NO: 10.

19. The promoter of claim 17, wherein the abiotic stress is cold stress.

20. The promoter of claim 17, wherein the abiotic stress is osmotic stress.
